(19) United States
(12) Patent Application Publication
(10) Pub. No.: US 2011/0186590 A1
(43) Pub. Date: Aug. 4, 2011

(54) STRIP ARRAYED STETHOSCOPE COVERS AND DISPENSING APPARATUS

(76) Inventor: Lanny Lee, Port St. Lucie, FL (US)

(21) Appl. No.: 13/019,955

(22) Filed: Feb. 2, 2011

Related U.S. Application Data
(63) Continuation-in-part of application No. 12/658,109, filed on Feb. 3, 2010, now abandoned.
Application No. 12/658,109 is a continuation-in-part of application No. 12/658,110, filed on Feb. 3, 2010.
Application No. 12/658,110 is a continuation-in-part of application No. 13/019,732, filed on Feb. 2, 2011.

(51) Int. Cl. B65H 5/28; A61B 19/02

(52) U.S. Cl. 221/73; 600/528

(57) ABSTRACT

A stethoscope cover and carrier strip assembly and a respective stethoscope diaphragm cover dispensing apparatus. The stethoscope cover and carrier strip assembly comprises a plurality of stethoscope covers detachably coupled to a carrier strip in a linear, spatial arrangement. Each cover is fabricated of a thin, elastically deformable membrane-like central portion defined by a peripheral edge. The covers are sized and shaped for placement over stethoscope diaphragm or a complete stethoscope head. The dispenser advances a cover into a staging area, where the cover is stretched in both a longitudinal and a lateral direction to allow unencumbered insertion of the stethoscope diaphragm or a complete stethoscope head. The cover is removed from the carrier and the spent carrier material is collected for disposition.

### RELATED US PATENT APPLICATION DATA

This patent application is a Continuation-In-Part Application, which claims priority to Non-Provisional application Ser. No. 13/019,732 filed on Feb. 2, 2011, which claims priority to Non-Provisional application Ser. No. 12/658,109 filed on Feb. 3, 2010 and Non-Provisional application Ser. No. 12/658,110, also filed on Feb. 3, 2010, which are both incorporated in their entireties by reference.

### FIELD OF THE INVENTION

The present disclosure generally relates to an apparatus and method of use for presentation of a sterile cover for a medical device. More particularly, the present disclosure relates to a dispensing apparatus that presents, stages by expansion, and singulates a sterile cover for a stethoscope head.

### BACKGROUND OF THE INVENTION

A stethoscope is a well known and widely used acoustic medical device for auscultation, or listening, to internal sounds in a human or animal body. The diaphragm is placed upon a patient, where body sounds vibrate the diaphragm creating acoustic pressure waves that travel through a tubing and are conveyed into a listener's ears. In use, the stethoscope head is often contaminated with skin bacteria and like contaminants that may undesirably be transmitted to the next patient. One means to avoid the undesirable transfer of unwanted organisms and other contaminants would be to sterilize the stethoscope head between each use. The transmission of bacterial infections among patients, particularly in a hospital setting, has been aggravated by the development of antibiotic-resistant strains of staphylococcal infections and other harmful microorganisms. It is this problem that the apparatus of the present invention seeks to overcome by providing a novel stethoscope cover and a dispensing apparatus for quickly and easily dispensing the stethoscope cover.

A number of different types of stethoscope covers and apparatus for dispensing the same have been suggested in the past.

A first exemplary proposed solution discloses stethoscope head cover that overlies the diaphragm and the surrounding rim of the stethoscope, the cover being fabricated from a thin sheet of plastic with a discontinuous layer of peelable adhesive on one surface of the plastic. The discontinuous layer of adhesive is in a predetermined pattern and the pattern is selected so that air passageways are provided from the regions between the cover and the diaphragm and past the rim to regions external the stethoscope. The cover generally comprises a flat, thin plastic sheet with the discontinuous layer of a peelable adhesive applied to one surface of the sheet. The adhesive adheres to both the diaphragm and the rim of the stethoscope leaving the aforesaid air passages for eliminating trapped air pockets or bubbles between the diaphragm and the cover.

A second exemplary disposable stethoscope cover diaphragm is removably attached over the outer edge of a stethoscope high frequency pickup in order to isolate the pickup from pathogens contacted when the stethoscope is used to examine a person. The cover diaphragm can be used as the diaphragm of a stethoscope pickup and can be used with a diaphragm supplied with the stethoscope. After the cover diaphragm has been used it can discarded and destroyed or, alternatively, can be cleaned for reuse.

An exemplary dispenser for use in dispensing disposable stethoscope diaphragms for removable attachment to a stethoscope head, supplies a series of diaphragms in a stacked array in a tube. The diaphragms are dispensed by a plate slidably mounted on the base for movement between retracted and extended positions. A cradle formed in the plate receives a single diaphragm through the opening when the dispensing plate is in its retracted position. Movement of the dispensing plate from its retracted to extended position is effective to place a diaphragm in the plate cradle into a position that allows the diaphragm to be attached to the stethoscope head by pressing the stethoscope head against the diaphragm.

What is desired is a disposable stethoscope diaphragm cover capable of being dispensed and presented for placement over a stethoscope head while minimizing any risk of contamination.

### SUMMARY OF THE INVENTION

This invention relates to stethoscope cover staging and dispensing apparatus, and more particularly to a stethoscope cover staging and dispensing apparatus which provides a medical professional an ability to place a sterile cover onto a stethoscope head.

In a first aspect of the present invention, a stethoscope cover and respective dispensing configuration, the stethoscope cover and respective dispensing configuration comprising:

a carrier strip fabricated of an elastically deformable material;

a plurality of stethoscope covers detachably coupled to the carrier strip in a linear, spatial arrangement, each cover being fabricated having a thin, elastically deformable membrane-like central portion defined by a peripheral edge;

wherein the plurality of stethoscope covers and carrier strip stretch in unison for staging and placement of each stethoscope cover onto one of a stethoscope head and a stethoscope diaphragm.

In a second aspect of the present invention, the carrier strip and plurality of stethoscope covers are fabricated of the same material during the same forming process.

In yet another aspect, the carrier strip and plurality of stethoscope covers are fabricated having a unitary structure.

In yet another aspect, each stethoscope cover is removably attached to the carrier strip by singulation webbing.

In yet another aspect, each stethoscope cover is removably attached to the carrier strip by a singulation groove formed about a periphery of the respective stethoscope cover.

In yet another aspect, the stethoscope covers are fabricated of a size and shape to cover a stethoscope head.

In yet another aspect, the stethoscope covers are fabricated of a size and shape to cover a diaphragm of a stethoscope head.

In yet another aspect, the stethoscope covers utilize an elastic property of the cover to attach to the entire stethoscope head or the diaphragm portion.

In yet another aspect, the carrier strip is rolled into a coil for distribution and dispensing.

In yet another aspect, the carrier strip is folded into a serpentine format for distribution and dispensing.

In yet another aspect, a stethoscope diaphragm cover dispensing apparatus inventories, feeds, and stages the stethoscope cover.

In yet another aspect, the carrier strip is drawn by either a manually operated advancing mechanism or an automated advancing mechanism.

In yet another aspect, the stethoscope diaphragm cover dispensing apparatus draws each of a first and a second carrier strip apart to expand the stethoscope cover during a staging step.

In yet another aspect, the stethoscope diaphragm cover dispensing apparatus draws each of a first and a second carrier strip outward, expanding the stethoscope cover into a staging configuration.

In yet another aspect, the stethoscope diaphragm cover dispensing apparatus comprises a cover clamping member, which temporarily retains the diaphragm cover in position during a step of inserting the stethoscope into the cover. This deters any contact with the cover to minimize any contamination prior to use.

In yet another aspect, the stethoscope diaphragm cover dispensing apparatus comprises a cover shearing mechanism which singulates the diaphragm cover from the carrier strip.

In yet another aspect, a method for individually staging a stethoscope cover from a series of stethoscope covers stored within a carrier strip, the method comprises the steps of:

providing a stethoscope cover and carrier strip assembly, the stethoscope cover and carrier strip assembly comprising:
a carrier strip fabricated of an elastically deformable material and
a plurality of stethoscope covers detachably coupled to the carrier strip in a linear, spatial arrangement, each cover being fabricated having a thin, elastically deformable membrane-like central portion defined by a peripheral edge,
wherein the plurality of stethoscope covers and carrier strip stretch in unison for staging and placement of each stethoscope cover onto one of a stethoscope head and a stethoscope diaphragm;

placing the stethoscope cover and carrier strip assembly into a stethoscope diaphragm cover dispensing apparatus;

advancing the stethoscope cover dispensing apparatus to position an individual stethoscope cover into a staging location;

stretching an opening of the stethoscope cover to allow insertion of at least a portion of a stethoscope head into the stethoscope cover;

simulating the stethoscope cover from the carrier strip; and

allowing the stethoscope cover to return towards a relaxed configuration, wherein the relaxed configuration secures the stethoscope cover to the respective portion of the stethoscope head.

In yet another aspect, the method further comprises the step of applying a longitudinal tension to the stethoscope cover and carrier strip assembly to expand the stethoscope cover in a longitudinal direction.

In yet another aspect, the method further comprises the step of applying a lateral tension to the stethoscope cover and carrier strip assembly to expand the stethoscope cover in a lateral direction.

An advantage of the invention allows the protective stethoscope head cover to be quickly and easily removed from the stethoscope head following examination of the patient and then suitably disposed of so as to prevent skin bacteria and like contaminants that may have contaminated the protective cover during patient examination from undesirably being transmitted to the next patient.

These and other advantages of the invention will be further understood and appreciated by those skilled in the art by reference to the following written specification, claims and appended drawings.

### BRIEF DESCRIPTION OF THE DRAWINGS

The invention will now be described, by way of example, with reference to the accompanying drawings, in which:

FIG. 1 presents an isometric view of an exemplary embodiment of a stethoscope diaphragm cover dispensing apparatus;

FIG. 2 presents a side elevation view of the exemplary stethoscope diaphragm cover dispensing apparatus, originally introduced in FIG. 1;

FIG. 3 presents a top plan view of the exemplary stethoscope diaphragm cover dispensing apparatus, originally introduced in FIG. 1, detailing an exemplary stethoscope diaphragm cover presentation configuration;

FIG. 4 presents a side elevation view of a second exemplary stethoscope diaphragm cover dispensing apparatus;

FIG. 5 presents a side elevation view of the second exemplary stethoscope diaphragm cover dispensing apparatus, originally introduced in FIG. 1, detailing a cover application process;

FIG. 6 presents a sectioned side elevation view of the second exemplary stethoscope diaphragm cover dispensing apparatus, originally introduced in FIG. 1, detailing a cover staging, the cover application process, and a cover singulation process;

FIG. 7 presents an isometric view of the operational components interacting with a stethoscope diaphragm cover array;

FIG. 8 presents a side view illustrating the stethoscope diaphragm cover as installed onto a stethoscope head;

FIG. 9 presents a sectioned side view taken across a central diameter of an alternate exemplary stethoscope diaphragm cover shown in a relaxed state;

FIG. 10 presents a sectioned side view taken across a central diameter of the stethoscope diaphragm cover of FIG. 8 shown in an extended, staging state;

FIG. 11 presents an isometric view of another exemplary stethoscope diaphragm cover dispensing apparatus for dispensing a stacked carrier strip.

Like reference numerals refer to like parts throughout the various views of the drawings.

### DETAILED DESCRIPTION OF THE INVENTION

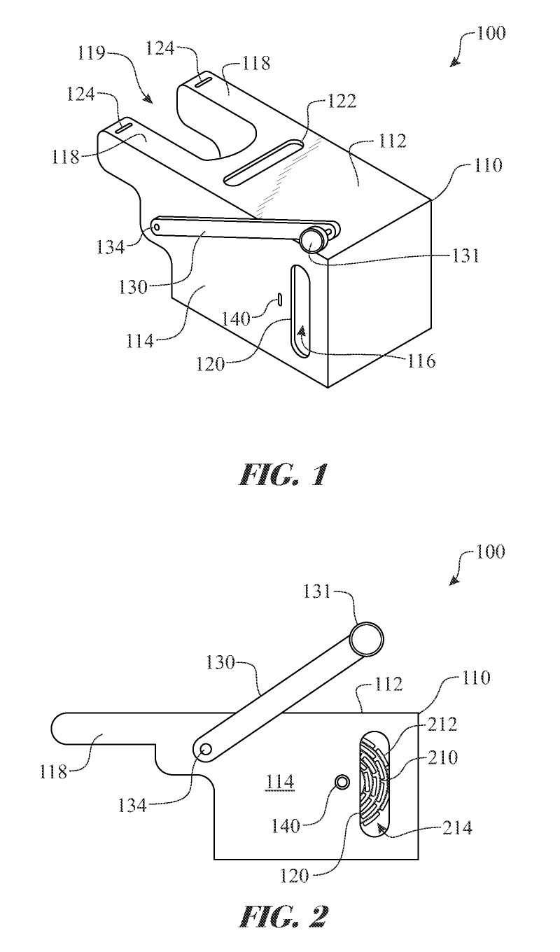

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

A stethoscope 300, as best illustrated in FIG. 8, is utilized in the medical profession for a multitude of applications. Sound can be utilized in diagnosing an ailment of a patient. The stethoscope 300 includes a stethoscope head 310 comprising at least one diaphragm 312, 314 which acquires and amplifies sounds emanating from within a patient. A pair of tubes in acoustical communication with the at least one diaphragm 312, 314, wherein the tubes transfer the amplified sounds to the medical professional's ears, audio equipment, and the like. The contact surface of the stethoscope head 310 often becomes contaminated with skin bacteria and like contaminants that must be disinfected to ensure against any undesirable transmission of the contaminants to the next patient. The present invention provides a stethoscope head cover 212 and a respective stethoscope diaphragm cover dispensing apparatus 100 to ensure the contacting surface of the stethoscope head 310 remains free from contaminants between patients.

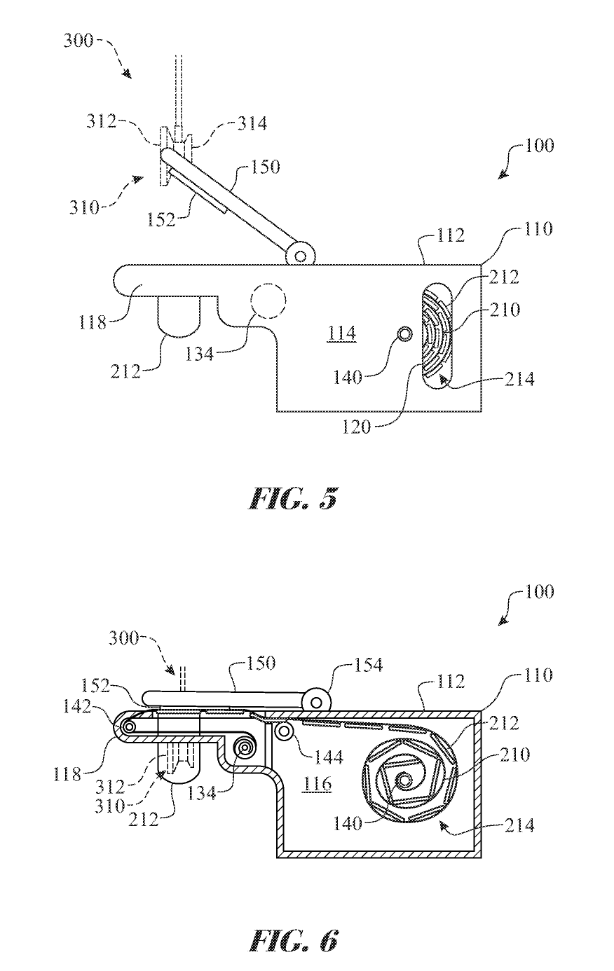

A stethoscope diaphragm cover dispensing apparatus 100 is provided for dispensing, staging, and placing a stethoscope head cover 212 onto a stethoscope head 310. Details of the stethoscope diaphragm cover dispensing apparatus 100 are presented in FIGS. 1 through 6, with the operational mechanisms being detailed in FIG. 8.

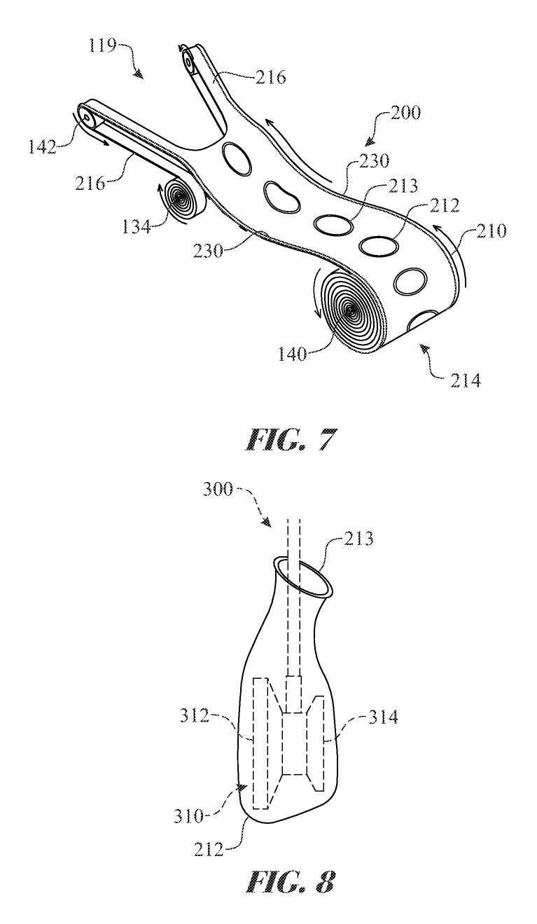

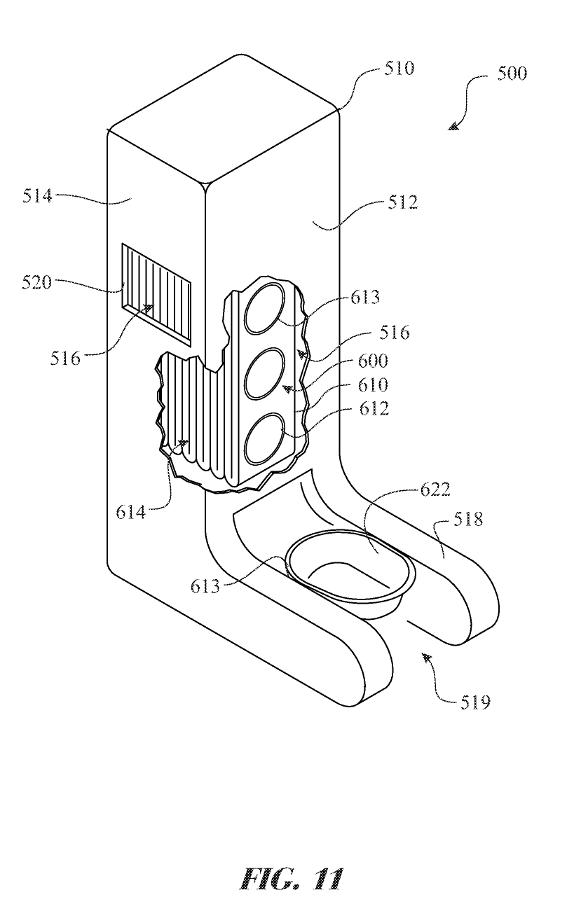

The stethoscope diaphragm cover dispensing apparatus 100 is designed to store, dispense, stage, and aid in a placement of a stethoscope head cover 212 onto a stethoscope head 310 as illustrated in FIGS. 3, 5, and 6. A 200 includes a series of stethoscope head covers 212, which are carried by a stethoscope carrier strip 210 as illustrated in FIGS. 7 through 9. Each stethoscope head cover 212 is fabricated having a thin, elastically deformable membrane-like central portion defined by a stethoscope head cover peripheral edge 213. The material selection ensures bacteria and other harmful microorganisms remain on the contacting (outer) surface of the stethoscope head cover 212. The stethoscope head cover 212 is formed having a size and shape to receive the stethoscope head 310. The interface between the stethoscope carrier strip 210 and the stethoscope head cover peripheral edge 213 can be defined to aid in separation of the stethoscope head cover 212 from the stethoscope carrier strip 210, but would be of a sufficient strength to allow the stethoscope carrier strip 210 to convey an expansion force onto the stethoscope head cover 212. This allows for the expansion of the stethoscope head cover 212 into an expanded stethoscope cover 222 for staging and insertion of the stethoscope head 310 thereinto. The stethoscope head cover 212 and the stethoscope carrier strip 210 can be formed simultaneously or separately and subsequently mated together. The stethoscope carrier strip 210 can be configured and stored in a rolled arranged referred to as a rolled carrier strip 214 and illustrated in FIGS. 6 and 7, or in a stacked serpentine configuration referred to as a serpentine carrier strip 614 and illustrated in FIG. 11.

The stethoscope diaphragm cover dispensing apparatus 100 includes a dispenser housing 110, which is fabricated having a pair of housing sidewalls 114 extending downward from each elongated edge of a housing top member 112 creating an inventory storage region 116 therein. The dispenser housing 110 is preferably fabricated of a sheet metal, plastic, a composite material, and the like. The stethoscope diaphragm cover dispensing apparatus 100 includes a pair of cover staging arms 118 extending forward from each outer edge of the dispenser housing 110 creating a cover staging clearance area 119. An upper surface of the cover staging arms 118 is preferably planar with the housing top member 112. A strip presentation aperture 122 can be provided through the housing top member 112 for passage of the stethoscope carrier strip 210 therethrough (as illustrated in FIG. 3). It is understood that the strip presentation aperture 122 can be alternatively located through a front member (not shown) of the dispenser housing 110. Alternatively, the strip presentation aperture 122 can be omitted, where the stethoscope carrier strip 210 would extend from underneath the housing top member 112. An excess strip material return port 124 is provided through an upper surface of each respective cover staging arm 118 for returning and collecting spent strip material 216 of the stethoscope cover and carrier strip assembly 200. The forward section of the housing top member 112 and the upper surfaces of each cover staging arm 118 can be used to create a platform for supporting the stethoscope head cover 212 during staging and placement upon the stethoscope head 310. An optional inventory viewing port 120 can be provided through any reasonable location of the dispenser housing 110 to determine the remaining inventory within the stethoscope diaphragm cover dispensing apparatus 100.

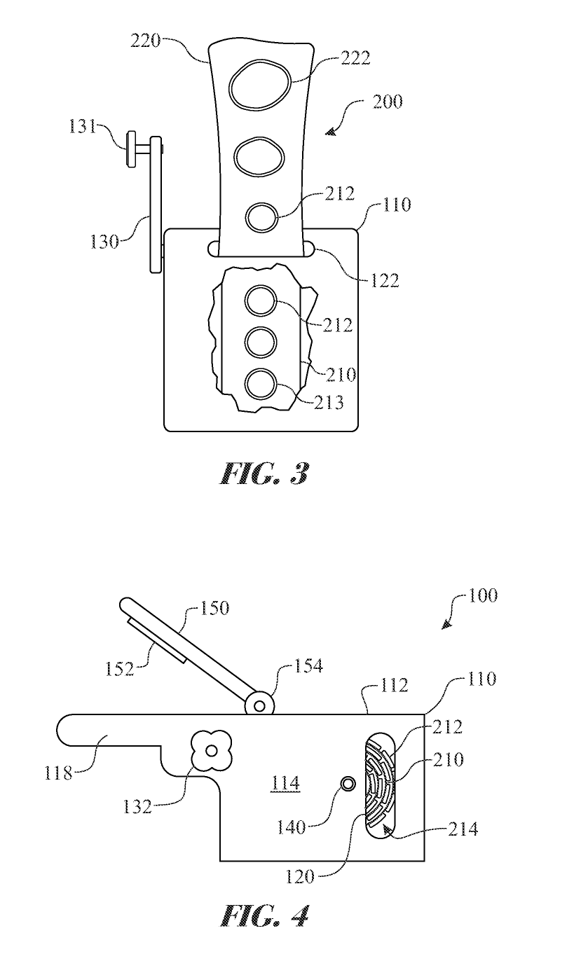

The internal operating mechanisms of the stethoscope diaphragm cover dispensing apparatus 100 are presented in FIGS. 6 and 7. The rolled carrier strip 214 is placed upon an inventory management axle 140 and inserted into the inventory storage region 116. The stethoscope carrier strip 210 is routed towards the cover staging clearance area 119. The rolled carrier strip 214 is preferably provided with a pair of leader strips, which emulate the spent strip material 216 and allow for preparation for use without wasting any stethoscope head covers 212. The spent strip material 216 is fed through each respective excess strip material return port 124, around a distal free wheel pulley 142, then returning and secured to a strip advance mechanism 134. The leader strip may include a feature to aid in engagement with the strip advance mechanism 134. Examples include a loop and pin or clip configuration, a slot within the strip advance mechanism 134, an adhesive, and the like. The strip advance mechanism 134 rotates to advance the stethoscope carrier strip 210. The strip advance mechanism 134 can be operated by any reasonable operating mechanism. In a first exemplary embodiment, a strip advancing control lever 130 (FIGS. 1 and 2) can operationally engage with the strip advance mechanism 134 using a ratcheting interface, wherein the medical professional would push/pull a lever handle 131 in a respective direction, causing the strip advancing control lever 130 to pivot accordingly. The pivotal motion of the strip advancing control lever 130 rotates the strip advance mechanism 134, which draws the stethoscope carrier strip 210 forward. A set of optional gears (not shown, but well understood) may be integrated between the strip advancing control lever 130 and the strip advance mechanism 134. The gear set can relate the pivotal direction of the strip advancing control lever 130 to the rotational direction of the strip advance mechanism 134 into either a like or an opposite direction. Additionally, the gear set can provide a torque conversion between the strip advancing control lever 130 and the strip advance mechanism 134.

An alternative exemplary mechanical drive means is a strip advancing control wheel 132 attached to the strip advance mechanism 134 in a manner similar to the engaging interface between the strip advancing control lever 130 and the strip advance mechanism 134 previously described herein. The medical professional would rotate the strip advancing control wheel 132 to rotate the strip advance mechanism 134, which draws the stethoscope carrier strip 210 forward.

An optional registration control system may be integrated into the stethoscope diaphragm cover dispensing apparatus 100. This can include a pin, a series of pins, and the like which engage with a notch, aperture, and the like spatially provided along the stethoscope carrier strip 210. The combination control the motion of the strip advance mechanism 134 ensuring the stethoscope head cover 212 is properly positioned within the cover staging clearance area 119 during the staging process.

It is understood that the advancing mechanism may be automated, integrating an electrically driven motor. At least one sensor can be integrated into the stethoscope diaphragm cover dispensing apparatus 100 to ensure proper registration of the stethoscope head cover 212 during the advancement process.

A tensioning member 144 is located rearward of the cover staging clearance area 119 and applies a longitudinal tension force to the stethoscope carrier strip 210. The tensioning member 144 illustrated is exemplary, where it is understood that tension may be applied by any reasonable mechanism. The excess strip material return ports 124 are positioned wider than a width between the edges of the stethoscope carrier strip 210 in a relaxed state. As the stethoscope carrier strip 210 is drawn into the excess strip material return port 124, the motion creates a lateral tensile force between the longitudinal edges of the stethoscope carrier strip 210. The tension applied to the stethoscope carrier strip 210 between the tensioning member 144 and the distal free wheel pulley 142 and by the distance between each of the excess strip material return ports 124 expands the stethoscope head cover peripheral edge 213 of the stethoscope head cover 212 into an expanded stethoscope cover 222. The expanded stethoscope cover 222 is a presented in a staged configuration of the stethoscope head cover 212 for insertion of the stethoscope head 310 thereinto.

An optional enlarged carrier edge 230 can be incorporated along each edge (or other portion) of the stethoscope carrier strip 210. The carrier rail feature or an enlarged carrier edge 230 can be any form factor having a thickness greater than the thickness of the stethoscope carrier strip 210, including a circular cross section, a “T” shaped cross section, an “L” shaped cross section, a “C” shaped cross section, and the like. The shape provides a mechanical interface to support the lateral tension. The enlarged carrier edge 230 would aid in providing the lateral tension to the stethoscope cover and carrier strip assembly 200. It is understood that the lateral stretching of the stethoscope cover and carrier strip assembly 200 can be created by any means of drawing each of a first edge and a second edge (understood by description) of the stethoscope carrier strip 210 apart from each other by positioning a first and a second distal draw location separated by a distance that is greater than a dimension between the a first edge and a second edge of the stethoscope carrier strip 210 when in a relaxed state.

An optional strip clamp 150 may be hingebly attached to the dispenser housing 110 by a strip clamp hinge 154. A cover shearing mechanism 152 may be integrated into a contacting surface of the strip clamp 150. The cover shearing mechanism 152 would be shaped to contour to an outline of the stethoscope head cover peripheral edge 213 respective to the stethoscope head cover 212 in an extended configuration and located in registration therewith. The medical professional advances the rolled carrier strip 214 positioning one stethoscope head cover 212 into a staging position within the cover staging clearance area 119 and subsequently inserts the stethoscope head 310 through the stretched stethoscope head cover peripheral edge 213 and into the expanded stethoscope cover 222. The medical professional can pivot the strip clamp 150 to temporarily hold the expanded stethoscope cover 222 in place during the insertion of the stethoscope head 310 thereinto. The medical professional can pivot the strip clamp 150 downward and applies a pressure thereon, which causes the cover shearing mechanism 152 to shear the stethoscope head cover 212 from the stethoscope carrier strip 210. Alternatively, the cover shearing mechanism 152 can be designed to apply a clamping force to the stethoscope carrier strip 210, where the stethoscope head cover 212 is separated from the stethoscope carrier strip 210 by applying a tensile force between the stethoscope head cover 212 and the stethoscope carrier strip 210 by moving the stethoscope head 310 accordingly. This provides an additional means to ensure the expanded stethoscope cover 222 remains untouched during placement onto the stethoscope head 310. The illustrated embodiment detailed in FIG. 8 places the stethoscope head cover 212 over the entire stethoscope head 310.

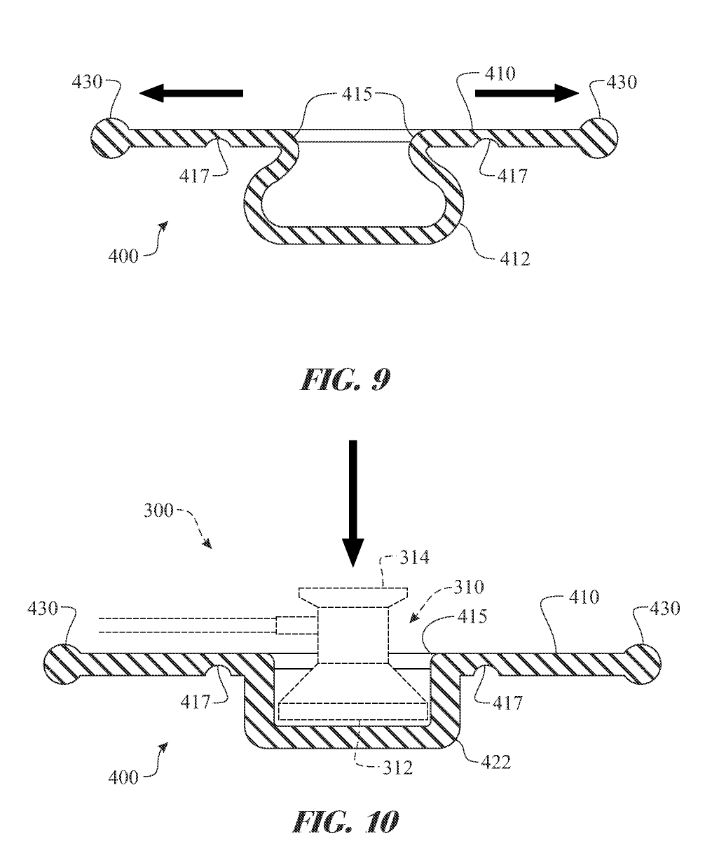

The stethoscope cover can be shaped in a second exemplary configuration referred to as a stethoscope diaphragm cover 412 and illustrated in FIGS. 9 and 10. The stethoscope diaphragm cover 412 includes a cover diaphragm engagement flange 415 formed about a peripheral edge. The stethoscope diaphragm cover 412 is carried by a stethoscope carrier strip 410 (collectively referred to as a stethoscope cover and carrier strip assembly 400) in a manner similar to that of the stethoscope head cover 212 and respective stethoscope carrier strip 210 previously described herein. The stethoscope diaphragm cover 412 is shaped to engage with and be retained upon a large diaphragm 312 or a small diaphragm 314 of the stethoscope head 310. The stethoscope diaphragm cover dispensing apparatus 100 draws the cover diaphragm engagement flange 415 radially outward allowing the medical professional to insert the diaphragm 312, 314 into the stethoscope diaphragm cover 412. A carrier rail feature or enlarged carrier edge 430 may be included along each of the distal edges of the stethoscope carrier strip 410 to mechanically aid in applying the lateral tension. The enlarged/shaped carrier edge 430 can be guided through a track, wherein the track is formed using any means known by those skilled in the art. The extended cover diaphragm engagement flange 415 retracts into a relaxed configuration when the stethoscope diaphragm cover 412 is separated from the stethoscope carrier strip 410. A cover singulation aid feature 417 may be included as a demarcation between the cover diaphragm engagement flange 415 and the stethoscope carrier strip 410. The cover singulation aid feature 417 can be designed to aid in the singulation process.

The above illustrates a stethoscope diaphragm cover dispensing apparatus 100 used in conjunction with the rolled carrier strip 214. Alternatively, a stethoscope diaphragm cover dispensing apparatus 500 may be used to dispense a stethoscope cover and carrier strip assembly 600 provided in a serpentine format. The stethoscope diaphragm cover dispensing apparatus 500 stores and dispenses a plurality of stethoscope head cover 612 from a serpentine carrier strip 614. Like features of stethoscope cover and carrier strip assembly 600 and stethoscope cover and carrier strip assembly 200 are numbered the same except preceded by the numeral ‘6’. A bead or other enlarged section of material may be provided about a stethoscope head cover edge 613 of the stethoscope head cover 612.

The stethoscope diaphragm cover dispensing apparatus 500 includes a dispenser housing 510, which can be formed having a pair of housing sidewalls 514 extending rearward from each elongated, vertical edge of a housing front member 512 creating an inventory storage section 516 therein. The dispenser housing 510 is preferably fabricated of a sheet metal, plastic, a composite material, and the like. The inventory storage section 516 is sized to receive the serpentine carrier strip 614. The stethoscope diaphragm cover dispensing apparatus 500 includes a pair of cover staging arms 518 extending forward from each outer edge of the dispenser housing 510 creating a cover staging clearance 519. The general conception and operational mechanisms are similar to those described for the stethoscope diaphragm cover dispensing apparatus 100. In the exemplary embodiment, the stethoscope carrier strip 610 is guided through a track or groove within an interior sidewall of each cover staging arm 518. An inventory viewing port 520 may be provided through the housing sidewall 514 or housing front member 512, allowing a service person to determine the remaining inventory within the inventory storage section 516. The stethoscope carrier strip 210 is drawn forward by any reasonable means. As the stethoscope carrier strip 210 is drawn forward, the stethoscope head cover 612 is expanded into a staged or expanded stethoscope cover 622 configuration, in accordance with the expansion process previously described herein.

It is understood that an optional protective layer may be adhered to at least one of the surfaces of the stethoscope cover and carrier strip assembly 200, 400, 600 to ensure sanitized delivery of the covers 212, 412, 612.

The stethoscope diaphragm cover dispensing apparatus 100, 500 can be provided in a variety of sizes and form factors, including a hand-held dispensing unit of type described that is of simple compact construction and one that can be inexpensively manufactured in quantity, a wall mounted unit, and the like. The system can include a manually advancing system, an automated advancing system, and the like.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What I claim is:

1. A stethoscope cover and carrier strip assembly, comprising:
a carrier strip fabricated of an elastically deformable material;
a plurality of stethoscope covers detachably coupled to said carrier strip in a linear, spatial arrangement, each cover being fabricated having a thin, elastically deformable membrane-like central portion defined by a peripheral edge;
wherein said plurality of stethoscope covers and carrier strip stretch in unison for staging and placement of each stethoscope cover onto one of a stethoscope head and a stethoscope diaphragm.

2. A stethoscope cover and carrier strip assembly as recited in claim 1, said plurality of stethoscope covers and said carrier strip are of a unitary construction.

3. A stethoscope cover and carrier strip assembly as recited in claim 1, said stethoscope cover and carrier strip assembly additionally comprising a bead formed about said peripheral edge.

4. A stethoscope cover and carrier strip assembly as recited in claim 1, said stethoscope cover and carrier strip assembly additionally comprising a cover singulation aid feature formed about said peripheral edge, wherein said cover singulation aid feature aids in separating each stethoscope cover from said carrier strip.

5. A stethoscope cover and carrier strip assembly as recited in claim 1, said stethoscope cover is sized and shaped to secure to said stethoscope diaphragm.

6. A stethoscope cover and carrier strip assembly as recited in claim 1, said stethoscope cover is sized and shaped to completely cover said stethoscope head.

7. A stethoscope diaphragm cover dispensing apparatus, comprising:
a strip advance mechanism to advance a stethoscope cover and carrier strip assembly, said stethoscope cover and carrier strip assembly comprising:
a carrier strip fabricated of an elastically deformable material;
a plurality of stethoscope covers detachably coupled to said carrier strip in a linear, spatial arrangement, each cover being fabricated having a thin, elastically deformable membrane-like central portion defined by a peripheral edge;
an inventory storage region configured to receive at least one of:
said stethoscope cover and carrier strip assembly arranged in a serpentine configuration, and
said stethoscope cover and carrier strip assembly arranged in a rolled configuration and further integrating an inventory management axle;
a staging mechanism which expands a staging stethoscope cover during said advancement by stretching said staging stethoscope cover in both lateral and longitudinal directions when advanced into a staging area, thus allowing insertion of one of a stethoscope head and a stethoscope diaphragm into said staging stethoscope cover.

8. A stethoscope diaphragm cover dispensing apparatus as recited in claim 7, wherein said strip advance mechanism draws said stethoscope cover and carrier strip assembly by collecting spent strip material.

9. A stethoscope diaphragm cover dispensing apparatus as recited in claim 8, wherein said longitudinal stretching is created by a tensioning member and said strip advance mechanism.

10. A stethoscope diaphragm cover dispensing apparatus as recited in claim 7, wherein said longitudinal stretching is created by a tensioning member located between said inventory storage region and said staging area.

11. A stethoscope diaphragm cover dispensing apparatus as recited in claim 7, wherein said lateral stretching is created by a drawing each of two edges of said carrier strip apart from each other.

12. A stethoscope diaphragm cover dispensing apparatus as recited in claim 7, wherein said lateral stretching is created by a drawing each of two edges of said carrier strip apart from each other utilizing a thickened edge feature located at least one of along and proximate each outer edge of said carrier tape.

13. A stethoscope diaphragm cover dispensing apparatus as recited in claim 7, wherein said lateral stretching is created by a drawing each of a first edge and a second edge of said carrier strip in a divergent manner by positioning a first and a second distal draw location separated by a distance that is greater than a dimension between said a first edge and a second edge of said carrier strip when in a relaxed state.

14. A stethoscope diaphragm cover dispensing apparatus, comprising:
an inventory storage region;
a strip advance mechanism to advance a stethoscope cover and carrier strip assembly, said stethoscope cover and carrier strip assembly comprising:
a carrier strip fabricated of an elastically deformable material;
a plurality of stethoscope covers detachably coupled to said carrier strip in a linear, spatial arrangement, each cover being fabricated having a thin, elastically deformable membrane-like central portion defined by a peripheral edge;
a staging mechanism which expands a staging stethoscope cover during said advancement by stretching said staging stethoscope cover in both lateral and longitudinal directions when advanced into a staging area, thus allowing insertion of one of a stethoscope head and a stethoscope diaphragm into said staging stethoscope cover.

15. A stethoscope diaphragm cover dispensing apparatus as recited in claim 14, wherein said strip advance mechanism draws said stethoscope cover and carrier strip assembly by collecting spent strip material.

16. A stethoscope diaphragm cover dispensing apparatus as recited in claim 15, wherein said longitudinal stretching is created by a tensioning member and said strip advance mechanism.

17. A stethoscope diaphragm cover dispensing apparatus as recited in claim 14, wherein said longitudinal stretching is created by a tensioning member located between said inventory storage region and said staging area.

18. A stethoscope diaphragm cover dispensing apparatus as recited in claim 14, wherein said lateral stretching is created by a drawing each of two edges of said carrier strip apart from each other.

19. A stethoscope diaphragm cover dispensing apparatus as recited in claim 14, wherein said lateral stretching is created by a drawing each of two edges of said carrier strip apart from each other utilizing a thickened edge feature located at least one of along and proximate each outer edge of said carrier tape.

20. A stethoscope diaphragm cover dispensing apparatus as recited in claim 14, wherein said lateral stretching is created by a drawing each of a first edge and a second edge of said carrier strip in a divergent manner by positioning a first and a second distal draw location separated by a distance that is greater than a dimension between said a first edge and a second edge of said carrier strip when in a relaxed state.
